(19) United States
(12) Patent Application Publication
(10) Pub. No.: US 2007/0038169 A1
(43) Pub. Date: Feb. 15, 2007

(54) Lower leg orthosis

(75) Inventors: Gad Alon, Rockville, MD (US); Mark S Hopkins, Baltimore, MD (US)

Correspondence Address: BARNES & THORNBURG LLP, P.O. BOX 2786, CHICAGO, IL, 60690-2786, US

(73) Assignee: Dankmeyer, Inc., Linthicum, MD (US)

(21) Appl. No.: 10/573,302

(22) PCT Filed: Jun. 30, 2004
(86) PCT No.: PCT/US04/22715

§ 371 Date: Sep. 7, 2006

Related U.S. Application Data
(60) Provisional application No. 60/504,430, filed on Sep. 22, 2003.

(51) Int. Cl. A61F 5/00

(52) U.S. Cl. 602/27

(57) ABSTRACT

An orthosis (10) for the lower leg of a wearer has a unitary body with a generally spiral shaped lower leg portion (14) and a foot plate portion (12). The orthosis is configured to allow simultaneous control of the knee and ankle-foot complex during locomotion.

### CROSS-REFERENCE TO RELATED APPLICATIONS

This application claims the benefit of the filing date of U.S. Provisional Application No. 60/504,430, filed on Sep. 22, 2003.

### BACKGROUND OF THE INVENTION

1. Field of the Invention

The present invention relates generally to orthotic devices for controlling the motion of a limb. More particularly, the present invention relates to an orthosis having a novel conformation that allows simultaneous control of the knee and ankle-foot complex during locomotion and a method of manufacturing the orthosis.

2. Background of the Technology

A large number of people suffer from ambulatory restrictions or limitations, which may be the result of natural or accidental causes. Among common causes of ambulatory problems in people is stroke. There are about 3 million people in the United States alone who are stroke survivors. Of that number about 20% are prescribed Ankle-Foot orthosis. About 50% of those have no control of both the knee and the ankle-foot complex. This number of approximately 300,000 patients have a need for an orthotic device that simultaneously can provide control of the knee and the ankle-foot complex. Each year there are approximately 700,000 new stroke survivors, thus adding approximately 70,000 new patients to the group in need of such an orthotic device. In addition, there are other hereditary or extraneous causal factors, such as cerebral palsy and traumatic brain injury, which can result in varying degrees of impairment of lower extremity neuro-motor performance.

It has been a long established practice to use orthotic devices to help support and control the movement of joints of the lower extremities. As discussed above, two common problems of the lower extremity that are suffered by patients are drop foot and hyper-extension of the knee.

When a person injures or loses motor control of their foot, orthotic devices are often used to brace and rehabilitate the foot. One such condition requiring the use of an orthotic device is when a person is unable to lift a foot, otherwise known as drop foot. Drop foot may be the result of brain damage from a stroke or head trauma, spinal injury, hereditary motor and sensory neuropathies, neuromuscular disease, or spinal disc impingement or pressure on nerves. Additionally, any damage to the muscle and nerves which activate the muscles of the neuromuscular system related to the foot may cause drop foot.

The neuromuscular system of the foot comprises the dorsiflexor muscles which are those on the front of the leg and below the knee which lift the foot. The action of lifting the foot from a position substantially aligned with the lower leg is known by the term dorsiflexion, while the action of extending the foot from a position substantially perpendicular to the lower leg to a position substantially aligned with the lower leg is known by the term plantar flexion. If the dorsiflexors are weak or the plantar flexors are hyperactive or both, a person will be unable to lift the foot as required in the swing phase of the step. The inability to lift the foot may cause a person to trip, since the foot may make contact with the ground during the downward swing of the leg.

In order to prevent drop foot a type of brace or orthosis known as an Ankle Foot Orthosis (AFO) has conventionally been used to compensate for weak dorsiflexors by resisting plantar flexion at heel strike and during swing phase of the step. Until the early 1960's, lower extremity bracing was essentially accomplished using metal, first steel and later the lighter aluminum in the construction of the AFO. In the late 1960's, plastic began to be used for orthotic devices, initially laminates and then, as in the case today, thermoplastics. The most prevalent AFO utilized is the posterior leaf-spring orthosis, which is typically fabricated by molding sheet plastic over a positive mold of a person's extremity. A posterior leaf-spring orthosis is typically a thermoplastic rigid 90° brace made to fit inside of a shoe. The mobility of an ankle held within the posterior leaf-spring orthosis is determined by the trim lines forming the shape of the orthosis and also by the thickness of the plastic. Another important component determining the mobility of the ankle is the attitude of the extremity when the cast was taken, i.e., dorsi-flexed, neutral, or planter-flexed, as well as the particular shoe to be worn.

U.S. Patent No. 5,370,604 issued to Bernardoni discloses an example of a posterior leaf-spring AFO, which assists a person suffering from drop foot, wherein the AFO is formed from a thermo-formed polypropylene material. Such AFO'S manufactured of unreinforced polypropylene did not provide sufficient dorsiflexion assistance because the materials were too flexible. Attempting to overcome the problem of over flexibility of polypropylene, some AFO's incorporated corrugations into their posterior surface and extending downward on their medial and lateral surfaces.

The posterior leaf-spring orthosis, has the limitation of restricting normal and desirable ankle and knee motion. For instance early attempts to prevent drop foot by increasing the dorsiflexion attitude of the orthosis also had the effect of making the knee joint unstable by inhibiting knee extension, without stopping the knee from hyper-extending during the stance phase. Without changing the attitude of the orthosis, the thickness of the posterior leaf-spring orthosis must be increased to provide the additional support to prevent drop foot. However, since the posterior leaf-spring orthosis fits within the shoe of the wearer, the thickness of the orthosis is limited by the available space between the wearer's foot and the shoe.

For the above reasons, alternative designs for an orthosis were developed which were superior to the posterior leaf-spring orthosis in that they prevented drop foot and controlled mild to moderate inversion and eversion, while still allowing more normal ankle and knee articulation. Such alternative designs include spiral and hemi-spiral AFO's. These and other alternative designs have also been manufactured from thermo-formable plastic material to maintain the light weight of the AFO. A recent example of a foot orthosis formed of copolymer thermoplastic materials is disclosed in U.S. Pat. No. 6,146,349 issued to Rothschild et al. Although more recent designs have had the benefit of being somewhat more functional and lighter, the support provided was still less than optimal, the knee and foot-ankle complex were not simultaneously treated with one device, and the selected construction materials were prone to failure and breakage. Once again, the conformation and the materials of the device did not provide the necessary support for the patient. Importantly, none of these orthosis provided relief for the patient who also suffered from hyper-extension of the knee.

Hyper-extension of the knee is another common problem of the lower extremities that can result from skeletal, ligament, neural, or muscular impairment or injury due to a multiple of factors including 1) if the ankle motion is not controlled 2) quadriceps is not controlled and/or 3) hip extensors control, such as gluteus maximus and hamstrings. Hyper-extension in stance is a common gait deficit that occurs when the quadriceps fail to perform their customary role during loading response and the first part of midstance. The quadriceps may not act appropriately in the event of quadriceps weakness, pain with quadriceps activation, or proprioceptive deficit.

In order to provide relief for the patient suffering from hyper-extension of the knee, the orthotic device is generally an elaborate multi-part orthosis, which requires considerable effort by the patient to put on and remove the device.

Such knee control devices are typically assemblies of multiple separate bands, heavy mechanical joints and securing straps that require time and considerable effort to secure to the patient's leg, making the patient dependent upon a care-giver to put on and remove the devices. Typically, the devices are also heavy and thick so as to be uncomfortable and unattractive for the wearer. U.S. Pat. No. 5,658,244 issued to Townsend et al. and U.S. Pat. No. 6,368,297 issued to Smits are representative of conventional orthosis use to treat knee injuries to include hyper-extension of the knee.

For both drop foot and hyper-extension of the knee, orthotic devices have been separately developed that have provided different levels of relief for the patient. A patient suffering from both conditions conventionally has been treated with separate orthotic devices. Conventional designed orthosis for control of hyper-extension of the knee or control of the ankle against drop foot are either designed as separate orthosis or, if combined, are designed as multiple component orthosis. These designs are heavy, bulky, do not allow transfer of mechanical energy, are non-cosmetic and usually require two hands to don and doff. Conventional designs limit the ability of the patient to tolerate the orthosis and frequently leads to skin irritation and pressure areas that in turn require repeat visits to realign the devices.

There remains therefore a need for a single unit, lower extremity orthotic device formed of a light-weight yet strong material conformed to allow simultaneous control of the knee and ankle-foot complex during locomotion.

### SUMMARY OF THE INVENTION

An embodiment of the present invention provides an orthosis that is a unitary structure that provides simultaneous control of the knee and the ankle-foot complex during locomotion.

An embodiment of the present invention provides an orthosis of unitary construction that is made that simultaneously controls both drop foot and knee hyper-extension.

An embodiment of the present invention provides an orthosis of unitary construction that transfers mechanical energy when worn by a walking subject and stops the knee from hyper-extending during the stance phase by generating a knee flexion moment or torque and simultaneously maintains the subjects foot in a position that allows the heel to contact the ground first at heel strike.

An embodiment of the present invention provides an orthosis of unitary construction that controls both drop foot and knee hyper-extension and can be worn with regular, off-the-shelf shoes or without any shoes.

A further embodiment of the present invention provides an orthosis of unitary construction for simultaneous control of the knee and the ankle-foot complex, being strong and light weight and fabricated of composite carbon fiber material.

An embodiment of the present invention provides an orthosis of unitary construction that can be securely worn by a subject without the need for straps or other added securing elements.

An embodiment of the present invention provides an orthosis of unitary construction that is light weight and can be easily domed and doffed by a subject using only one hand.

An embodiment of the present invention provides a method for controlling the movement of the ankle-foot complex in a subject in need of treatment for drop foot.

An embodiment of the present invention provides a method of treating a subject in need of treatment for knee hyper-extension during locomotion.

An embodiment of the present invention provides a method of simultaneously treating a subject in need of treatment for drop foot and knee hyper-extension.

These and other aspects of the present invention will become apparent from the following description when taken in connection with the accompanying drawings which, for purposes of illustration only, show embodiments in accordance with the present invention.

### BRIEF DESCRIPTION OF THE DRAWINGS

This invention will be explained further in detail with reference to the accompanying drawings in which:

FIG. 1 is a lateral to medial side view of a device configured according to an embodiment of the invention;

FIG. 2 is a medial to lateral side view of the device of FIG. 1;

FIG. 3 is anterior to posterior view of the device of FIG. 1;

FIG. 4 is a posterior to anterior view of the device of FIG. 1;

FIG. 5 is a diagrammatic side view of the device of FIG. 1 as worn on the lower leg of a subject; and

FIG. 6 is a diagram showing the orthosis posting angle and relative dimensions of a device configured according to an embodiment of the invention.

### DETAILED DESCRIPTION OF THE INVENTION

The present invention relates to an orthosis having a novel conformation that allows simultaneous control of the knee and ankle-foot complex during locomotion.

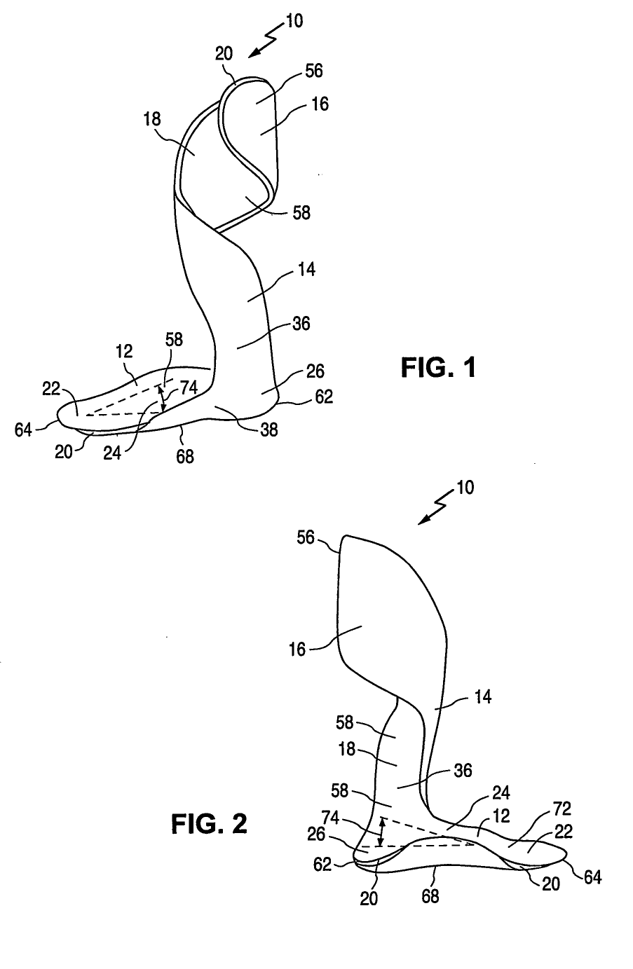

As shown in FIGS. 1-5, the orthosis, generally shown at 10, is a single structure having a foot portion 12, which transitions into an ascending, spiraling lower leg portion 14. The orthosis generally has an outer surface 16 and an inner surface 18, which come into contact one with the other at an edge surface 20, which follows the contours of the orthosis 10.

The foot plate portion 12 includes an anterior section 22, a middle section 24, and a posterior section 26, which are configured to generally accept the shape of a wearer's foot 30. The heel 28 of the wearer's foot 30 contacts the inner surface 18 of the posterior section 26 and the toes 32 of the wearer's foot contacts the inner surface 18 of the anterior section 22. The foot plate portion 12 can be generally contoured to comfortably complement at least a portion and preferably all of the plantar surface 34 of the wearer's foot 30 so as to permit the wearer to wear shoes if desired while also wearing the orthosis 10. The foot plate portion 12 also includes a substantially flat outer surface 16. The fit of the orthosis 10 on the foot is such that it can be worn with normal shoes, specially fitted shoes, sandals of any style or no shoes without discomfort or unwanted shifting of position of the orthosis 10 on the foot.

The ascending, spiraling lower leg portion 14 originates as an ascending section 36, which extends from the lateral aspect 38 of the middle section 24 of the foot plate portion 12. The ascending extension 36 is initially disposed on at least a portion of the lateral surface 40 of the lower leg 42 of a wearer. Above the ankle 44 of the wearer the ascending section 36 curves toward the anterior surface 46 of the lower leg 42 to form a shin section 48 of the lower leg portion 14. The shin section 48 covers at least a portion of the anterior surface 46 of the lower leg 42 and continues to spiral around at least a portion of the medial surface 50 (indicated by dotted line) of the lower leg 42 as it transitions into a posteriorly disposed calf section 52 of the lower leg portion 14. The calf section 52 continues to spiral around the lower leg 42 so as to cover at least a portion of the posterior surface 54 of the lower leg 42 of the wearer. The calf section 52 terminates in an upper end 56 of the lower leg portion 14 at a point where the calf section 52 begins to spiral back toward the anterior surface 46 of the lower leg 42.

As described above, the lower leg portion 14 is uniquely configured to spiral upward from the middle section 24 of the foot plate portion 12 in a novel manner, first to the anterior surface 44 of the lower leg 42 of the wearer and, as it ascends, then to the posterior surface 54 of the lower leg 42 of the wearer. The angle or degree of ascension of the spiraling lower leg portion 14 can gradually decrease as the lower leg portion 14 nears the calf section 52 of the lower leg 42 of the wearer.

This novel configuration of the orthosis 10 of the present invention has the effect of transferring mechanical energy during walking so as to simultaneously stop the knee from hyper-extending during the stance phase by generating a knee flexion moment (torque) while maintaining the foot in a position that allows the heel to contact the ground first at heel strike. The dynamics of the orthosis 10 during locomotion also minimizes medio-lateral joint instability. In general, the novel configuration of the orthosis 10 of the present invention performs such that during loading, knee extension and ankle plantar flexion transfer a force that stresses the upper leg portion 14, the hemi-spiral, and uses the potential to kinetic energy transfer to resist knee extension and thus eliminate hyper-extension while providing strong support for the ankle-foot complex.

The present invention provides this simultaneous support for the knee and ankle-foot complex in a single, light-weight unit without the need for multiple components, jointed elements, straps, bands or any additional attachments or securing means that are normally required in traditional orthosis. Indeed, the simultaneous treatment of knee hyper-extension and drop foot is conventionally accomplished using a separate orthosis for each infirmity and those devices used are typically constructed of many heavy, bulky components that require considerable dexterity and effort to don and doff. The novel configuration of the present invention facilitates the easy putting on and taking off of the orthosis 10 by a wearer using only one hand. This additional benefit of the present invention over a conventional orthosis is particularly important to stroke survivors or other subjects who for any reason do not have the full use of both of their upper extremities.

The orthosis 10 of the present invention can be manufactured using unitary construction methods of light weight materials having sufficient strength to provide the needed support to the knee and foot-ankle complex while facilitating the transfer of mechanical energy generated during locomotion to create a dorsiflexion moment (torque) while maintaining the foot in a position that allows the heel to contact the ground first at heel strike. One material that can be used for construction of the orthosis of the present invention is composite carbon fiber material. Other construction materials which can provide the light-weight, strong support required by the present invention can also be employed. The inner surface 18 of the orthosis 10 can be fixedly or removably covered by a lining material 58 that provides a soft, durable, protective surface between the strong, light-weight material of the orthosis 10 and the skin of the wearer. If such a lining material 58 is fixedly attached to the inner surface 18, of the orthosis 10, such attachment can be accomplished using any conventional mechanical or chemical means known. If such a lining material 58 is removably attached to the orthosis 10, such removable attachment can be by the use of releasable chemical or electrostatic adhesive, hook and loop structures such as Velcro™, or any other known releasable attachment means. It is within the concept of the present invention to be able to provide the orthosis 10 without a lining material 58.

Manufacture of the orthosis 10 of the present invention can be accomplished by any mechanical or hand molding or material forming process known. The unitary construction provides a light-weight device having good mechanical energy transfer qualities that serve to provide simultaneous control for the knee and the foot-ankle complex. The orthosis 10 can be custom made to fit the needs of the individual wearer or can be manufactured by mass production as an off-the-shelf device that is available in predetermined sizes, alignments, and foot plate dimensions. In manufacturing the orthosis, 10, the edge surface 20 can be shaped or contoured to provide a smooth or generally rounded surface to improve the comfort level and ease of use experienced by the wearer during use or during the putting-on or taking-off of the orthosis 10. The edge surface 20 can also be treated, coated, or covered with a protective material or chemical surface so as to provide a non-abrasive contact to skin or clothing.

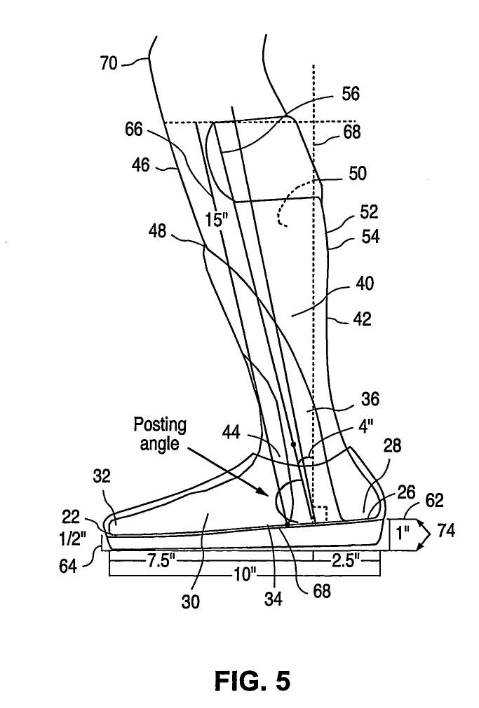

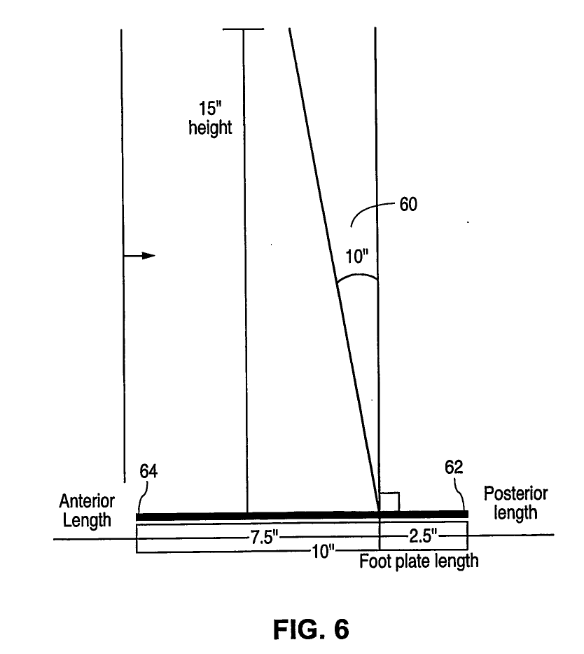

The function of the system is affected by several factors, including: the material stiffness of the orthosis 10, the novel configuration of the spiral design of the lower leg portion 14, the design of the foot plate portion 12, the proximal and distal lever arms length (as best shown in FIG. 5 and FIG. 6 at 66 and 68, the orthosis posting angle 60, the heel height (heel-toes differential) as demonstrated in FIG. 6 as the difference between the back edge 62 and the front edge 64, and the shoe posting as best shown in FIG. 5.

In forming the orthosis 10, the orthosis posting angle 60; that is, the angle or degree of inclination from the vertical that is created by the alignment of the foot plate portion 12 to the lower leg portion 14 can have a bias from posterior to anterior as best shown in FIGS. 5 and 6. This orthosis posting angle 60 can be adjusted as necessary in the manufacture of a custom-fitted orthosis to meet the specific needs of the wearer. In some embodiments, the orthosis posting angle 60 will be between about 7 degrees and about 14 degrees of forward or anterior inclination from the vertical relationship of the foot plate portion 12 and the lower leg portion 14. The range of the orthosis posting angle 60 may also be between about 9 degrees and about 12 degrees. In one embodiment, the posting angle 60 is about 10 degrees from the vertical, as shown in FIG. 6.

The lower leg portion 14 of the orthosis can be formed to begin its upward ascent from the foot plate portion 12 at a point measured from the back edge 62 of the posterior section 26 that is approximately ¼ of the total length of the foot plate portion 12. Forward or backward variance from this upward ascent position of the lower leg portion 14 can be allowed without departing from the scope of the present invention.

The foot plate portion 12 can be configured to have a general forward slant relative to the ground or surface on which the wearer is standing. This general forward slant of the foot plate portion 12 can be accomplished by configuring the orthosis such that the back edge 62 of the foot plate portion 12 is elevated above the level of the front edge 64 of the foot plate portion 26 in multiples of approximately two; that, is for example, an orthosis that provides a foot plate portion configured to having the back edge 62 elevated above the ground or floor level by approximately one inch will have the front edge 64 elevated above the ground or floor level by approximately one-half inch. This non-binding example of the present invention having such a slant for the foot plate portion 12 is best shown in FIG. 5. A variance to this embodiment wherein the elevation relationship between the back edge 62 and the front edge 64 is approximately two can be permitted without departing from the scope of the present invention.

FIG. 5 and FIG. 6 provide an example diagrammatic representation of the orthosis 10 of the present invention. As recited above and shown in FIG. 5 and FIG. 6, the dimensions and angles recited for this non-limiting example are for demonstration purposes.

An embodiment of the orthosis 10 includes the configuration of the foot plate portion 12 having the outer surface 16 of the foot plate portion 12 substantially flat. This configuration facilitates achieving optimal force and pressure distribution with the contact surface of the shoe/sandal (footwear) or with the floor/ground, if the orthosis 12 is worn without footwear. In an embodiment of the invention, the length of the foot plate 12 extends the along the length of the foot 30 up to the toes so as to provide a long distal lever arm 68. Similarly, the highest reach of the upper end 56 of the orthosis 10 on the lower leg 42 of the wearer should be below the knee 70 of the wearer so as to provide an appropriate length of the orthosis 10 along the longitudinal axis of the lower leg 42. The calf section of the lower leg portion 52 of the orthosis 10 must be as large as possible in proportion to the size of the wear's calf so as to optimize the mechanical energy transfer while minimizing the amount of pressure (pressure=force/surface area) imposed by the calf section 52 on the posterior surface of the lower leg 54 of the wearer. As best shown in FIGS. 5 and 6, an embodiment of the present invention has long proximal and distal lever arms 66, 68, a large foot contact surface 72, and a large calf section 52 to generate optimum force and torque to eliminate knee hyper-extension.

The orthosis posting angle 60 is the angle created between the longitudinal axis of the foot plate portion 12 and the longitudinal axis of the lower leg portion 14 of orthosis 10. In some embodiments, the posting angle 60 is between 10 degrees and 15 degrees, which eliminates knee hyper-extension for a large number of wearers. For some wearers the preferred posting angle 60 may be 20° to 25° and for others a posting angle 60 of only 0-5° would be preferred to optimize control of the ankle/foot and knee joints. An inadequate posting angle 60 can result in a poor transfer of mechanical energy and thus provide an inadequate level of control of the knee.

Foot plate posting is another aspect of the invention that contributes to the optimization of forces and torque generation between the orthosis 10 and the limb of the wearer and thereby further facilitates control of the knee, ankle, and foot. Foot plate posting is particularly important if the orthosis 10 is worn without a shoe/sandal (footwear).

The sagittal plane posting angle 74, as best shown in FIGS. 2 and 5, refers to the angle formed by the elevation of the posterior section 26 relative to the anterior section 22 of the foot plate portion 12. The height differential between the posterior section 26 and the anterior section 22 can range between 0.0 cm to 3.0 cm. For a large number of wearers, the preferred height differential to form the sagittal plane posting angle 74 is approximately 1.7 cm to 2.3 cm and approximately 2.0 cm is most preferred. For other wearers, the preferred height differential to form the sagittal plane posting angle is approximately 1.0 cm to 1.5 cm. For still other wearers, the preferred height differential to form the sagittal plane angle is approximately 2.5 cm to 3.0 cm.

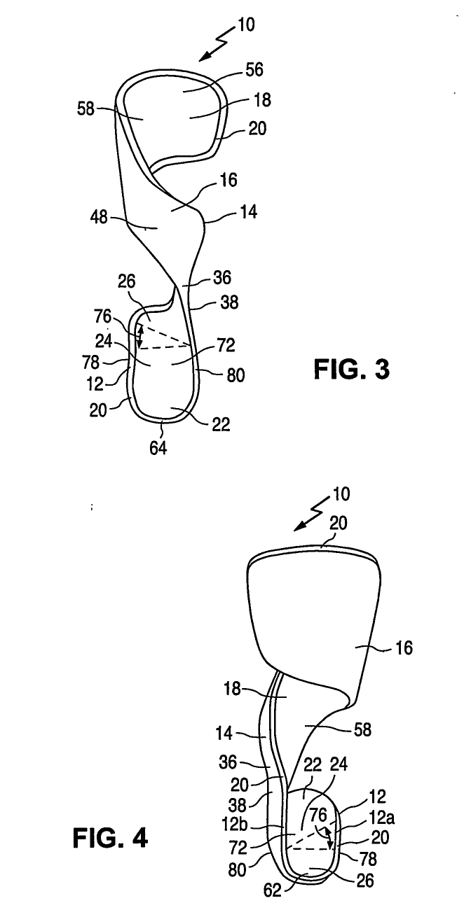

The frontal plane posting angle 76, as best shown in FIGS. 3 and 4, refers to the angle formed by the elevation of the lateral side 78 of the foot plate 12 relative to the medial side 80 of the foot plate 12. The frontal plane posting angle 76 refers to and is important for controlling foot-ankle positioning in the frontal plane (medio-lateral control). The height differential between the lateral side 78 and the medial side 80 can range between 0.0 to 10.0 mm. For a large number of wearers, the most preferred height differential to form the frontal plane posting angle 76 is approximately 2.0 mm to 3.0 mm. For other wearers, the preferred height differential to form the frontal plane posting angle is approximately 5.0 mm to 7.0 mm. For still other wearers, who require more medio-lateral control at the margins of the ranges offered by the orthosis 10, the preferred height differential to form the frontal plane angle can be approximately 0.0 mm to 1.0 mm or at the other extreme, 8.0 mm to 10 mm.

If the orthosis 10 is used with footwear (shoe/sandal), the posting angle 60 can be applied to the footwear, and thus to a lesser degree, be an important feature similar to the posting angle 60 of the orthosis 10. Similarly, when the orthosis 10 is worn with footwear, the toe-heel elevation differences and the medial-lateral elevation differences of the footwear can effect the sagittal plane posting angle and the frontal plane posting angle effect on the wearer. For this reason, to optimize the corrective results for the wearer of the orthosis 10, configuration of the orthosis 10 and the method of treatment of the wearer should include a thorough study and evaluation of the wearer's anatomical features as well as the mode of use (with or without footwear) and, if footwear is used with the orthosis 10, the specific configuration of that footwear should be carefully considered.

A method by which a patient is fitted for the various embodiments of the orthosis described herein will now be described.

Evaluation: A health care professional (such as a physician, nurse, or physician's assistant) interviews the patient to determine history of present illness, past medical history, social history, including current and previous orthosis use and desired functional goals. The health care professional conducts a physical examination to include observation, range of motion, strength and motor control, skin integrity/edema, sensory testing, palpation and soft tissue assessment, observational gait analysis and functional tests. The health care professional also presents (to the patient) options and plans for the intended orthosis, including the use and care of the orthosis, the impact on daily activities and footwear.

Casting and measuring: The patient is positioned in sitting with the knee at 90 degrees and the foot positioned on a pre-fabricated footplate with a heel differential of ⅜ inches. The leg is measured and measurements recorded as follows: two circumferential calf measurements, two linear medio-lateral measurements of the calf and ankle, two height measurements of the fibular neck and medial malleolus and a foot length measurement. The limb is then prepared with two layers of nylon casting stockinette and a latex tubing down the anterior aspect of the leg. Boney landmarks are defined on the stockinette with indelible ink pencil: medial and lateral malleolus, first and fifth metatarsal heads and fifth metatarsal base, navicular tuberosity, fibular neck and any other pertinent prominences. The limb is wrapped with plaster or synthetic casting material positioned in 10 degrees of ankle DF as measured from a reference of the footplate. The material is allowed to set, cut down the anterior aspect over the tubing and removed from the patient. Portion of the material removed from the patient is referred to in future steps as the cast.

A method by which various embodiments of the orthosis described herein are fabricated will now be described.

Cast Preparation: The cast is sealed and reinforced with additional plaster bandage. A release agent is applied to the inside of the cast.

Positive Model Fabrication: The cast is filled with liquid plaster and a pipe mandrel is inserted into the plaster one inch from the bottom with eight inches extending from the top and held in this position until the plaster is set. The cast is then stripped from the positive plaster model.

Positive Model Rectification and Preparation: The positive model is cleaned and the indelible pencil marks, which have transferred from the inside of the cast, are reinforced. The model is then rectified to relieve for the lateral malleolus, fifth metatarsal base and tibial crest. The plantar surface is flattened, and a flare is added the edges of the model in the intended spiral pattern.

Orthosis Lamination, Trim Out and Finish: The positive model is sealed with lacquer. Interface padding material is then pulled over the spiral section of the model. The model and padding is then sealed in a polyvinyl chloride (PVA) bag under suction. A lay up of carbon fiber braid, carbon tape and nyglass is pulled over the model in a specific and detailed manner for proper strength and flexibility. A second PVA bag is pulled over the entire unit. Liquid acrylic resin is applied to the lay up inside of the bag and suction is applied. The plastic is allowed to cure. When cured, the spiral pattern is cut out and the rough shape pulled off of the plaster model. The edges are buffed and cleaned and the interface padding installed.

Patient Fitting: The orthosis is then fit to the patient, adjusted for comfort. Basic instruction in use and care is provided including donning and doffing, cleaning and storage.

While the invention has been set forth in great detail, it is appreciated by persons skilled in the art that the orthosis may be subject to various modifications and rearrangements without departing from the scope of the invention.

What is claimed is:

1. An orthosis comprising a single, unitary body made of composite carbon fiber, the unitary body comprising:
a generally spiral-shaped lower leg portion that, when the orthosis is worn, covers at least a portion of the anterior surface of the wearer's lower leg and at least a portion of the posterior surface of the wearer's lower leg; and
a foot plate portion that, when the orthosis is worn, accepts the wearer's foot.

2. The orthosis of claim 1, wherein the foot plate portion comprises:
an anterior section; and
a posterior section,
wherein, when the orthosis is worn, the wearer's toes contact the anterior section and the wearer's heel contacts the posterior section.

3. The orthosis of claim 1, wherein:
the unitary body transfers mechanical energy while the wearer is walking;
the unitary body uses the transferred mechanical energy to stop the wearer's knee from hyperextending; and
the unitary body maintains the wearer's foot in a position that allows the heel of the person's foot to contact the ground before the rest of the wearer's foot.

4. The orthosis of claim 1, wherein the lower leg portion comprises an ascending section that extends from the foot plate section and curves toward the anterior surface of the wearer's lower leg to form a shin section.

5. The orthosis of claim 4, wherein, from the shin section, the lower leg portion continues to spiral around at a least a portion of a lateral surface of the wearer's lower leg and transitions into a calf section.

6. The orthosis of claim 5, wherein the calf section terminates in an upper end of the lower leg portion and begins to spiral back toward the anterior surface of the wearer's lower leg.

7. The orthosis of claim 1, wherein the lower leg portion is configured to spiral upward from the foot plate portion and make contact with an anterior surface of the wearer's lower leg prior to making contact with a posterior surface of the wearer's lower leg as the lower leg portion ascends.

8. The orthosis of claim 7, wherein the angle at which the lower leg portion spirals upward gradually decreases as the lower leg portion nears the calf of the wearer.

9. An orthosis comprising a single, unitary body, the unitary body comprising:
a generally spiral-shaped lower leg portion that, when the device is worn, covers at least a portion of the anterior surface of the wearer's lower leg and at least a portion of the posterior surface of the wearer's lower leg; and
a foot plate portion that, when the device is worn, accepts the wearer's foot,
wherein the lower leg portion is configured to spiral upward and clockwise from the foot plate portion and, as it ascends, to make contact with an anterior surface of the wearer's lower leg prior to making contact with a posterior surface of the wearer's leg.

10. The orthosis of claim 9, wherein the foot plate portion comprises:
an anterior section; and
a posterior section,
wherein, when the orthosis is worn, the wearer's toes contact the anterior section and the wearer's heel contacts the posterior section.

11. The orthosis of claim 9, wherein:
the unitary body transfers mechanical energy while the person is walking;
the unitary body uses the transferred mechanical energy to stop the person's knee from hyperextending; and
the unitary body maintains the person's foot in a position that allows the heel of the person's foot to contact the ground before the rest of the person's foot.

12. The orthosis of claim 9, wherein the lower leg portion comprises an ascending section that extends from the foot plate section and curves toward the anterior surface of the wearer's lower leg to form a shin section.

13. The orthosis of claim 12, wherein, from the shin section, the lower leg portion continues to spiral around at a least a portion of a lateral surface of the wearer's lower leg and transitions into a calf section.

14. The orthosis of claim 13, wherein the calf section terminates in an upper end of the lower leg portion and begins to spiral back toward an anterior surface of the wearer's lower leg.

15. The orthosis of claim 9, wherein the lower leg portion is configured to spiral upward from the foot plate portion and make contact with an anterior surface of the wearer's lower leg prior to making contact with a posterior surface of the wearer's lower leg as the lower leg portion ascends.

16. The orthosis of claim 15, wherein the angle at which the lower leg portion spirals upward gradually decreases as the lower leg portion nears the calf of the wearer.

17. The orthosis of one of claims 1-16, wherein the foot plate portion is generally contoured to complement at least a portion of the plantar surface of the wearer's foot so as to permit the wearer to wear shoes or sandals.

18. The orthosis of one of claims 1-16, wherein the unitary body comprises:
an inner surface; and
a lining material attached to the inner surface.

19. The orthosis of claim 18, wherein the lining material is removably attached to the inner surface.

20. The orthosis of claim 18, wherein the lining material is removably attached to the inner surface using Velcro™.

21. The orthosis of one of claims 1-16, wherein the unitary body is made of composite carbon fiber.

22. The orthosis of one of claims 1-16, wherein the lower leg portion comprises a section that extends from the foot plate section.

23. The orthosis of claim 22,
wherein the foot plate section comprises a posterior section, and
wherein the section that extends from the foot plate section is disposed at about ¼ of the length of the foot plate section from the posterior section.

24. The orthosis of one of claims 1-16, wherein the lower leg portion comprises an ascending section that extends from the foot plate section and curves toward the anterior surface of the wearer's lower leg to form a shin section.

25. The orthosis of claim 24, wherein, from the shin section, the lower leg portion continues to spiral around at a least a portion of a lateral surface of the wearer's lower leg and transitions into a calf section.

26. The orthosis of claim 25, wherein the calf section terminates in an upper end of the lower leg portion and begins to spiral back toward the anterior surface of the wearer's lower leg.

27. The orthosis of one of claims 1-16,
wherein the foot plate portion and the lower leg portion are disposed at an angle with respect to one another, and
wherein the difference between the angle and 90 degrees is between about 7 degrees and about 14 degrees.

28. The orthosis of one of claims 1-16,
wherein the foot plate portion and the lower leg portion are disposed at an angle with respect to one another, and
wherein the difference between the angle and 90 degrees is between about 9 degrees and about 12 degrees.

29. The orthosis of one of claims 1-16,
wherein the foot plate portion and the lower leg portion are disposed at an angle with respect to one another, and
wherein the difference between the angle and 90 degrees is about 10 degrees.

30. The orthosis of one of claims 1-16,
wherein the foot plate portion and the lower leg portion are disposed at an angle with respect to one another, and
wherein the difference between the angle and 90 degrees is between about 20 degrees and about 25 degrees.

31. The orthosis of one of claims 1-16,
wherein the foot plate portion and the lower leg portion are disposed at an angle with respect to one another, and
wherein the difference between the angle and 90 degrees is between about 9 degrees and about 12 degrees.

32. The orthosis of one of claims 1-16,
wherein the foot plate portion comprises a front edge and a back edge,
wherein the back edge is elevated above the front edge.

33. The orthosis of one of claims 1-16,
wherein the foot plate portion comprises a front edge and a back edge,
wherein the distance between the back edge and ground level is about twice the distance between the front edge and ground level.

34. The orthosis of one of claims 1-16,
wherein the foot plate portion comprises a front edge and a back edge,
wherein the distance between the back edge and ground level is about 1 inch and the distance between the front edge and ground level is about ½ inch.

35. The orthosis of one of claims 1-16,
wherein the foot plate portion comprises an anterior section and a posterior section,
wherein the difference in elevation between the anterior section and the posterior section is greater than 0 centimeters and no greater than about 3 centimeters.

36. The orthosis of one of claims 1-16,
wherein the foot plate portion comprises an anterior section and a posterior section,
wherein the difference in elevation between the anterior section and the posterior section is between about 1.7 centimeters and about 2.3 centimeters.

37. The orthosis of one of claims 1-16,
wherein the foot plate portion comprises an anterior section and a posterior section,
wherein the difference in elevation between the anterior section and the posterior section is about 2.

38. The orthosis of one of claims 1-16,
wherein the foot plate portion comprises an anterior section and a posterior section,
wherein the difference in elevation between the anterior section and the posterior section is between about 1 centimeter and about 1.5 centimeters.

39. The orthosis of one of claims 1-16,
wherein the foot plate portion comprises an anterior section and a posterior section,
wherein the difference in elevation between the anterior section and the posterior section is between about 2.5 centimeters and about 3 centimeters.

40. The orthosis of one of claims 1-16,
wherein the foot plate portion comprises a medial side and a lateral side,
wherein the difference in elevation between the medial side and the lateral side is greater than 0 millimeters and no greater than about 10 millimeters.

41. The orthosis of one of claims 1-16,
wherein the foot plate portion comprises a medial side and a lateral side,
wherein the difference in elevation between the medial side and the lateral side is between about 2 millimeters and about 3 millimeters.

42. The orthosis of one of claims 1-16,
wherein the foot plate portion comprises a medial side and a lateral side,
wherein the difference in elevation between the medial side and the lateral side is between about 5 millimeters and about 7 millimeters.

43. The orthosis of one of claims 1-16,
wherein the foot plate portion comprises a medial side and a lateral side,
wherein the difference in elevation between the medial side and the lateral side is between about 8 millimeters and about 10 millimeters.

44. The orthosis of one of claims 1-16,
wherein the foot plate portion comprises a medial side and a lateral side,
wherein the difference in elevation between the medial side and the lateral side is greater than 0 millimeters and no greater than about 1 millimeter.

45. The orthosis of one of claims 1-16, wherein the foot plate portion has a substantially flat outer surface.
